(19) United States
(12) Patent Application Publication
(10) Pub. No.: US 2024/0358261 A1
(43) Pub. Date: Oct. 31, 2024

(54) CERVICAL BODY TEMPERATURE CHANGE MONITORING SYSTEM AND METHOD

(71) Applicant: PUKYONG NATIONAL UNIVERSITY INDUSTRY-UNIVERSITY COOPERATION FOUNDATION, Busan (KR)

(72) Inventors: Byeong Il LEE, Busan (KR); Myung Gi YI, Busan (KR)

(73) Assignee: PUKYONG NATIONAL UNIVERSITY INDUSTRY-UNIVERSITY COOPERATION FOUNDATION, Busan (KR)

(21) Appl. No.: 18/646,836

(22) Filed: Apr. 26, 2024

(30) Foreign Application Priority Data
Apr. 27, 2023 (KR) 10-2023-0055758

(51) Int. Cl. A61B 5/01; A61B 5/00

(52) U.S. Cl. CPC A61B 5/015; A61B 5/7264

(57) ABSTRACT

The present disclosure relates to a cervical body temperature change monitoring system and method, in which a cervical body temperature change can be monitored by measuring body temperatures of the cervical vertebra before and after treatment and analyzing the measurement results. A cervical body temperature change monitoring system may include: a cervical vertebral area processing unit that determines whether the cervical vertebra is recognized using at least one pressure sensor and performs segmentation on cervical vertebra recognition area to generate a plurality of segmented areas; a temperature distribution analysis unit that measures a body temperature in the cervical vertebra recognition area using at least one temperature sensor and generates a temperature distribution map; and a body temperature change measurement unit that derives a treatment effect by using the map.

### CROSS-REFERENCE TO RELATED APPLICATION

This application claims priority to and the benefit of Korean Patent Application No. 10-2023-0055758 filed on Apr. 27, 2023, in the Korean Intellectual Property Office, the entire disclosure of which is incorporated herein by reference.

### BACKGROUND

### 1. Field

The present disclosure relates to a cervical body temperature change monitoring system and method. More specifically, the present disclosure relates to a cervical body temperature change monitoring system and method, in which a cervical body temperature change can be monitored by measuring body temperatures of the cervical vertebra before and after treatment and analyzing the measurement results.

### 2. Description of the Related Art

Recently, the market for personal healthcare devices to continuously monitor an individual's health has been growing explosively. The healthcare devices encompass wearable devices represented by smart watches. In addition, there are also health management pillows for monitoring an individual's sleeping condition, etc.

When a patient receives treatment at a medical institution such as an oriental medicine clinic or an orthopedic clinic, the treatment is generally performed while lying or in a prone position on a bed. Treatment at such a medical institution generally has the effect of relaxing body muscles or facilitating blood circulation. The effects of the aforementioned treatment are difficult to show as standardized results, and there is a difficulty in confirming the effectiveness of the above-mentioned treatment by receiving feedback on how a patient feels after the treatment is completed.

Korean Patent No. 10-1519469 (May 6, 2015) discloses a conventional health monitoring pillow.

### SUMMARY

The present disclosure provides a cervical body temperature change monitoring system and method, in which, in which a cervical body temperature change can be monitored by measuring body temperatures of the cervical vertebra before and after treatment and analyzing the measurement results.

A cervical body temperature change monitoring system according to an embodiment of the present disclosure may include: a cervical vertebral area processing unit that determines whether the cervical vertebra is recognized using at least one pressure sensor and performs segmentation on cervical vertebra recognition area to generate a plurality of segmented areas; a temperature distribution analysis unit that measures a body temperature in the cervical vertebra recognition area using at least one temperature sensor and generates a temperature distribution map using the measurement results; and a body temperature change measurement unit that derives a treatment effect by using the temperature distribution map.

The cervical vertebral area processing unit may include: a cervical vertebra recognition determination module that determines whether the cervical vertebra is recognized using the pressure sensor; and an area segmentation module that, when the user's cervical vertebra is recognized, generates at least two segmented areas by performing segmentation on the cervical vertebra recognition area, which is an area in which the cervical vertebra is recognized.

The segmented areas may be formed by using a preset number of or a preset reference wideness of segmented areas, and each of the segmented areas may be formed to include at least one temperature sensor.

The temperature distribution analysis unit may include: a body temperature information acquisition module that acquires body temperature distribution information as a measurement result of the body temperature using the temperature sensor provided in the cervical vertebra recognition area; and a temperature distribution map generation module that generates the temperature distribution map for the cervical vertebra recognition area using the segmented areas and the body temperature distribution information.

The temperature distribution map may be generated using the segmented areas and the body temperature distribution information, and may include a definition for each of the segmented areas through machine learning.

The body temperature change measurement unit may include: a segmented area profile generation module that cumulatively stores the temperature distribution map to generate a profile for each segmented area; and a treatment effect analysis module that derives the treatment effect by analyzing the profile for each segmented area.

The profile for each segmented area may be generated by using the temperature distribution map from the moment the body temperature is first measured to the moment the treatment is completed.

The treatment effect may be derived by deriving the change in the body temperature for each of the segmented areas and comparing the derived change in the body temperature with a preset reference.

A cervical body temperature change monitoring method according to an embodiment of the present disclosure may include: a cervical vertebral area processing step of a cervical vertebral area processing unit determining whether the cervical vertebra is recognized using at least one pressure sensor and performing segmentation on a cervical vertebra recognition area to generate a plurality of segmented areas; a temperature distribution analysis performing step of a temperature distribution analysis unit measuring a body temperature in the cervical vertebra recognition area using at least one temperature sensor and generating a temperature distribution map using the measurement results; and a body temperature change measurement performing step of a body temperature change measurement unit deriving a treatment effect by using the temperature distribution map.

The cervical vertebral area processing step may include: determining whether the cervical vertebra is recognized using the pressure sensor; and when the user's cervical vertebra is recognized, generating at least two segmented areas by performing segmentation on the cervical vertebra recognition area, which is an area in which the cervical vertebra is recognized.

The segmented areas may be formed by using a preset number of or a preset reference wideness of segmented areas, and each of the segmented areas may be formed to include at least one temperature sensor.

The temperature distribution analysis performing step may include: acquiring body temperature distribution information as a measurement result of the body temperature using the temperature sensor provided in the cervical vertebra recognition area; and generating the temperature distribution map for the cervical vertebra recognition area using the segmented areas and the body temperature distribution information.

The temperature distribution map may be generated using the segmented areas and the body temperature distribution information, and may include a definition for each of the segmented areas through machine learning.

The body temperature change measurement performing step may include: cumulatively storing the temperature distribution map to generate a profile for each segmented area; and deriving the treatment effect by analyzing the profile for each segmented area.

The profile for each segmented area may be generated by using the temperature distribution map from the moment the body temperature is first measured to the moment the treatment is completed.

The treatment effect may be derived by deriving the change in the body temperature for each of the segmented areas and comparing the derived change in the body temperature with a preset reference.

### BRIEF DESCRIPTION OF DRAWINGS

FIG. 1 is a block diagram of a cervical body temperature change monitoring system according to an embodiment of the present disclosure.

FIG. 2 is a block diagram of a cervical vertebral area processing unit in the cervical body temperature change monitoring system of FIG. 1.

FIG. 3 is a block diagram of a temperature distribution analysis unit in the cervical body temperature change monitoring system of FIG. 1.

FIG. 4 is a block diagram of a body temperature change measurement unit in the cervical body temperature change monitoring system of FIG. 1.

FIG. 5 is a flowchart showing a cervical body temperature change monitoring method according to an embodiment of the present disclosure.

FIG. 6 is a flowchart of step S11 in the cervical body temperature change monitoring method shown in FIG. 5.

FIG. 7 is a flowchart of step S13 in the cervical body temperature change monitoring method shown in FIG. 5.

FIG. 8 is a flowchart of step S15 in the cervical body temperature change monitoring method shown in FIG. 5.

FIG. 9 shows a schematic diagram of a system for implementing one or more aspects of the present disclosure.

### DETAILED DESCRIPTION

Hereinafter, embodiments of the present disclosure are described in detail with reference to illustrative drawings. In various different drawings, the same components are designed with the same or similar reference numerals and detailed descriptions thereof may be omitted. In addition, as to known configurations or functions, reference is made to known technologies and detailed descriptions thereof may be omitted. Throughout the specification, when the terms “include”, “comprise”, “have”, “consist of”, etc. are used, unless “only ˜” is used, other components may be added. When a component is expressed in the singular form, plural forms may also be included, unless clearly indicated otherwise.

In addition, in describing the components of the present disclosure, terms such as first, second, A, B, (a), and (b) may be used. These terms are only used to distinguish the components from other components, and the natures, sequences, orders, or numbers of the components are not limited by these terms.

In describing the positional relationship of components, when two or more components are described as being “linked,” “coupled,” or “connected,” etc., two or more components may be directly “linked,” “coupled,” or “connected,” but it should be understood that other components different from the two or more components may be further “interposed” to be “linked,” “coupled,” or “connected”. Here, the other components may also be included in one or more of the two or more components that are “linked,” “coupled,” or “connected” to each other.

In explaining temporal flow relationships related to components, operation methods, manufacturing methods, etc., for example, temporal precedence relationships such as “after ˜”, “subsequent to ˜”, “following ˜”, “before ˜”, etc. or in describing sequential relationships, unless the term “immediately” or “directly” is used, cases of being non-continuous may also be included.

Meanwhile, when numerical values or corresponding information about a component (e.g. level, etc.) are mentioned, unless separately explicitly stated, the numerical values or the information corresponding thereto can be interpreted as including error ranges that may occur due to various factors (e.g., process factors, internal or external shocks, noises, etc.).

In addition, the technical features described in the cervical body temperature change monitoring system can also be applied to the cervical body temperature change monitoring method, and the reverse is also true.

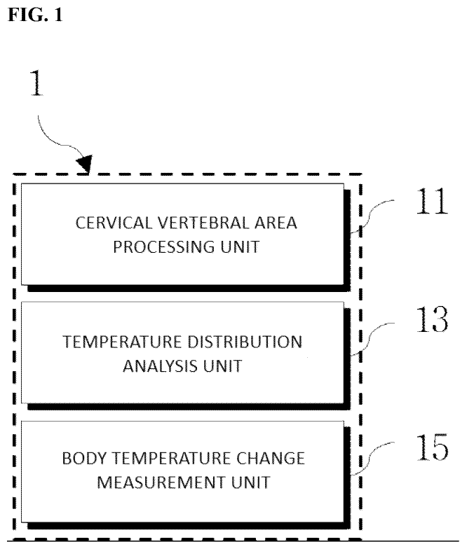

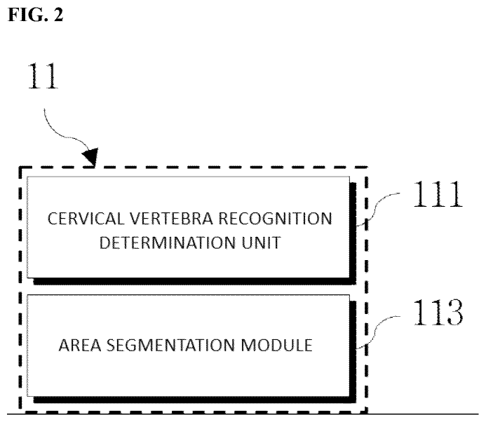

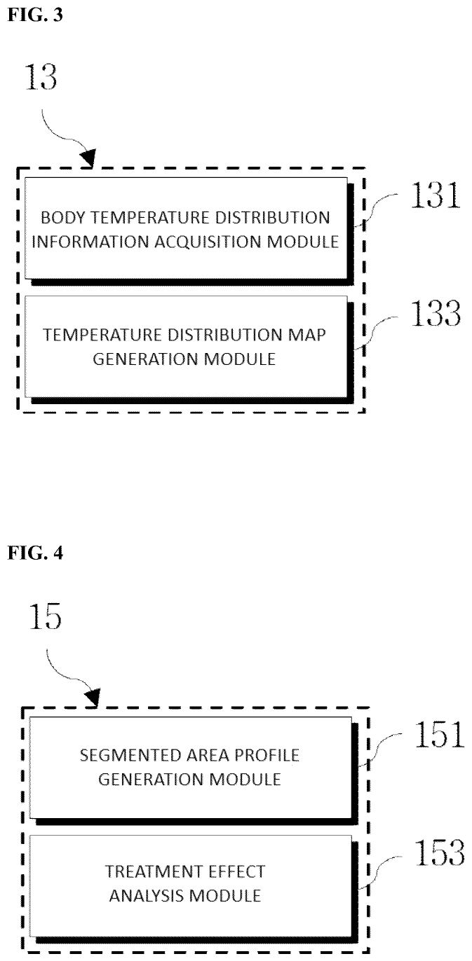

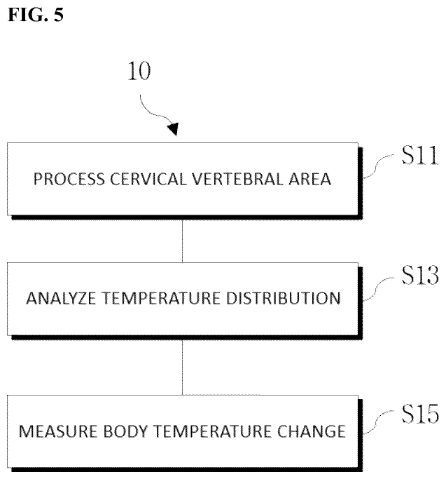

FIG. 1 is a block diagram of a cervical body temperature change monitoring system 1 according to an embodiment of the present disclosure, FIG. 2 is a block diagram of a cervical vertebral area processing unit, FIG. 3 is a block diagram of a temperature distribution analysis unit, and FIG. 4 is a block diagram of a body temperature change measurement unit.

Hereinafter, referring to FIGS. 1 to 4, a cervical body temperature change monitoring system (for brevity, to be referred to as a monitoring system, hereinafter) according to an embodiment of the present disclosure will be described in detail.

Referring to the drawings, the monitoring system 1 is configured to measure the body temperature on a cervical vertebral area of a user (treatment subject), generate a temperature distribution map using the measured body temperature, and monitor a user's body temperature change by cumulatively acquiring and analyzing the generated temperature distribution map before treatment and after treatment. To this end, as shown in FIG. 1, the monitoring system 1 may include a cervical vertebral area processing unit 11, a temperature distribution analysis unit 13, and a body temperature change measurement unit 15.

The cervical vertebral area processing unit 11 may be configured to determine whether the cervical vertebra is recognized using a pressure sensor and generate a plurality of segmented areas. The cervical vertebral area processing unit 11 may determine whether the user's cervical vertebra is recognized by using at least one pressure sensor, and, when the user's cervical vertebra is recognized as a result of the determination, identifies a cervical vertebra recognition area and segments the identified area to generate a plurality of segmented areas. To this end, the cervical vertebral area processing unit 11 according to an embodiment of the present disclosure may include a cervical vertebra recognition determination module 111 and an area segmentation module 113, as shown in FIG. 2.

The cervical vertebra recognition determination module 111 may be configured to determine whether the cervical vertebra is recognized by using a pressure sensor. Here, the pressure sensor may include at least one pressure sensor, and preferably a plurality of pressure sensors may be formed in an N×M matrix. This is for performing area segmentation by means of the area segmentation module 113, which will be described later, in order to generate a temperature distribution map in the present disclosure.

Even if only one pressure sensor is used, body temperature changes in the user's cervical vertebral area can be monitored, but a plurality of pressure sensors may be used to obtain more accurate monitoring results by measuring body temperature changes according to the location of the cervical vertebral area.

The cervical vertebra recognition determination module 111 may determine that the user's cervical vertebral area is recognized when a pressure greater than a preset level is measured through a pressure sensor. In another embodiment, the cervical vertebra recognition determination module 111 may be set to determine that the user's cervical vertebral area is recognized when a temperature in a preset temperature range is measured using a temperature sensor included in the monitoring system 1 of the present disclosure. Here, by recognizing the cervical vertebral area using both a temperature sensor and a pressure sensor, the recognition accuracy of the cervical vertebral area can be improved.

As another example, if a temperature in a preset range is measured using only a temperature sensor without a pressure sensor, it may be determined that the cervical vertebral area is recognized. In this case, the cervical vertebral area can be recognized even without a separate pressure sensor, thereby forming a simplified system or a system with reduced costs.

In the present embodiment, if the cervical vertebra recognition determination module 111 determines that the user's cervical vertebral area is recognized, the area segmentation module 113 may be configured to perform segmentation on a cervical vertebra recognition area, which is an area where the cervical vertebra is recognized. The area segmentation module 113 may acquire the area where the cervical vertebra is recognized using a pressure sensor, and may be configured to generate at least two segmented areas by segmenting a pertinent area.

Here, the segmented areas may be formed by using a preset number of or a preset reference wideness of segmented areas, and each of the segmented areas may be formed to include at least one temperature sensor. By using this, the monitoring system 1 of the present disclosure may obtain temperature changes in the respective segmented areas.

When a plurality of segmented areas are obtained through segmentation of the cervical vertebra recognition area, the temperature distribution analysis unit 13 is configured to measure the body temperature of the cervical vertebra recognition area using at least one temperature sensor and generate a temperature distribution map using the measurement result. To this end, as shown in FIG. 3, the temperature distribution analysis unit 13 according to an embodiment of the present disclosure may be configured to include a body temperature information acquisition module 131 and a temperature distribution map generation module 133.

The body temperature information acquisition module 131 may be configured to acquire body temperature distribution information, which is a measurement result of the user's body temperature using a temperature sensor provided in the cervical vertebra recognition area. The body temperature information acquisition module 131 may be configured to acquire body temperature using a temperature sensor. Here, the temperature sensor may be provided as at least one included in each of the segmented areas, as described above, and by using this, in one embodiment of the present disclosure, the body temperature information acquisition module 131 may obtain body temperature distribution information, which is a measurement result of the body temperature for each of the segmented areas.

The temperature distribution map generation module 133 may be configured to generate a temperature distribution map for the cervical vertebra recognition area using the segmented areas and body temperature distribution information. The temperature distribution map is generated using the segmented areas, and is configured to express the body temperature distribution information of the entire cervical vertebra recognition area in two dimensions using the body temperature distribution information measured in each of the segmented areas.

Meanwhile, the temperature distribution map generation module 133 may perform defining on each of the segmented areas using machine learning. When the user's cervical vertebra is recognized, the temperature distribution map generation module 133 according to an embodiment of the present disclosure may be configured to acquire the distribution of measured pressure values and use the same as learning data, while defining the positions of segmented areas constituting the cervical vertebra recognition area of a current user by applying the previously obtained distribution of pressure values of other users to a preset algorithm.

In this case, the pressure values may be mapped to a specific location in the cervical vertebral area according to the distribution thereof. Therefore, the temperature of a specific location in the cervical vertebral area can be known, and a treatment effect can be derived depending on the temperature and/or temperature change at the location. In this case, the treatment effect can be more accurately analyzed.

When the generation of the temperature distribution map is completed, the body temperature change measurement unit 15 may be configured to derive a treatment effect by using the temperature distribution map. To this end, the body temperature change measurement unit 15 may be configured to include a segmented area profile generation module 151 and a treatment effect analysis module 153, as shown in FIG. 4.

The segmented area profile generation module 151 may be configured to generate a profile for each segmented area by cumulatively storing the temperature distribution map. Here, the profile for each segmented area may cumulatively store temperature distribution maps, and thus may include time-series information. The profile for each segmented area may be generated including at least the temperature distribution map before treatment and the temperature distribution map after treatment. More preferably, the profile for each segmented area may be generated including all temperature distribution maps that are cumulatively stored at a preset cycle using the body temperature measured from the moment the recognition of the user's cervical vertebra is performed until just before the recognition of the user's cervical vertebra is completed.

The treatment effect analysis module 153 may be configured to derive a treatment effect by analyzing the profile for each segmented area. Here, the treatment effect may be derived by deriving a body temperature change for each of the segmented areas and comparing the derived body temperature change with a preset reference. In an embodiment of the present disclosure, the treatment effect analysis module 153 may select a reference temperature distribution map among a plurality of temperature distribution maps included in the profile for each segmented area to derive the body temperature changes, and may obtain the amount of temperature change in each segmented area as time-series information based on the temperature of the selected standard temperature distribution map.

In addition, the treatment effect analysis module 153 may be configured to compare the obtained treatment effect and an expected effect to further derive a personal treatment effect for an actual pertinent user. The treatment effect analysis module 153 may obtain the expected effect corresponding to a treatment method that is pre-entered by a treatment practitioner and compare the obtained expected effect with the treatment effect to derive how effective the treatment method actually is for a pertinent user. Here, the actual effect can be expressed as a percentage, which is the ratio of the treatment effect to the expected effect.

The treatment effect analysis module 153 may be configured to derive treatment effects using, in addition to time-series information, a method for analyzing other profiles for the segmented areas. To derive a treatment effect for a specific portion, the treatment effect analysis module 153 may derive the treatment effect by comparing the left and right profiles of a cervical vertebral area for each segmented area, and, to determine how effective the personal treatment effect for the pertinent user is, the treatment effect analysis module 153 may perform a comparison with other users' profiles for each segmented area.

Here, the treatment effect analysis module 153 may relatively determine the extent of the effect of the treatment provided to the pertinent user by cumulatively storing the treatment effects for other users, and may identify and provide an optimal treatment method using the profiles of other users showing similar treatment effects for each segmented area.

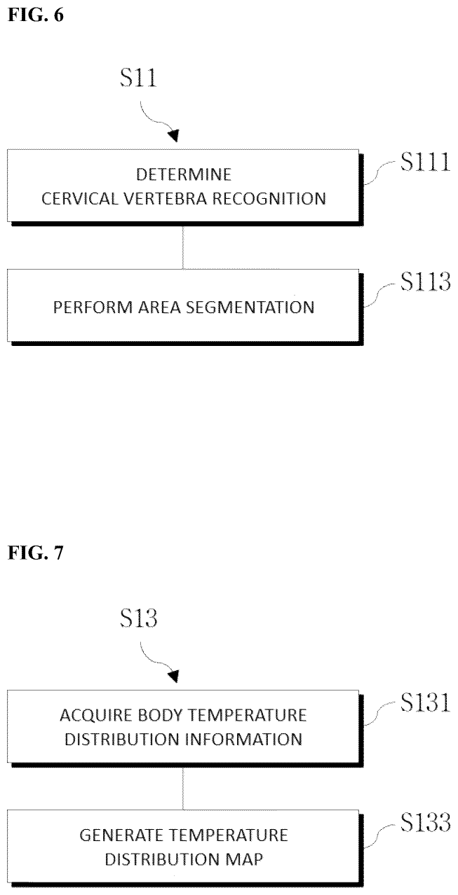

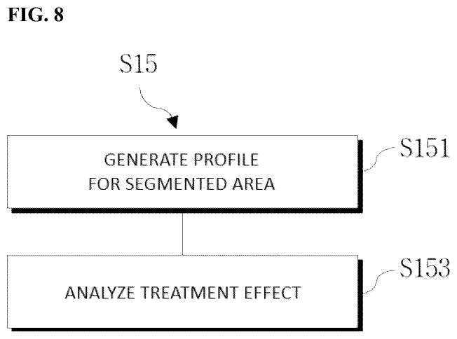

Meanwhile, FIGS. 5 to 8 show a cervical body temperature change monitoring method according to an embodiment of the present disclosure. FIG. 5 is a flowchart showing a cervical body temperature change monitoring method according to an embodiment of the present disclosure, FIG. 6 is a flowchart of step S11, FIG. 7 is a flowchart of step S13, and FIG. 8 is a flowchart of step S15.

Hereinafter, the cervical body temperature change monitoring method of the present disclosure (hereinafter, for brevity, referred to as the monitoring method) will be described. In addition, for convenience of explanation, the method of the present disclosure is described as using the above-described system of FIG. 1, but the present disclosure is not necessarily used in the system of FIG. 1.

The monitoring method 10 according to an embodiment of the present disclosure is configured to measure the body temperature of the cervical vertebral area of a user (a treatment subject), generate a temperature distribution map using the measured body temperature, and monitor user's body temperature changes by cumulatively acquiring temperature distribution maps generated from before treatment to after treatment. To this end, as shown in FIG. 5, the monitoring method 10 according to an embodiment of the present disclosure may include: a cervical vertebral area processing step (S11), a temperature distribution analysis performing step (S13), and a body temperature change measurement performing step (S15).

The cervical vertebral area processing step (S11) may be configured to determine whether the cervical vertebra is recognized using a pressure sensor and generate a plurality of segmented areas, in a cervical vertebral area processing unit. In the cervical vertebral area processing step (S11), it is determined whether the user's cervical vertebra is recognized using at least one pressure sensor, and if the user's cervical vertebra is recognized as a determination result, the cervical vertebra recognition area is identified, and a plurality of segmented areas are generated by segmenting the identified area. To this end, as shown in FIG. 6, the cervical vertebral area processing step (S11) may be configured to include determining whether the cervical vertebra is recognized (S111) and performing area segmentation (S113).

The step of determining whether the cervical vertebra is recognized (S111) may be configured to determine whether the cervical vertebra is recognized by using a pressure sensor. Here, the pressure sensor may include at least one pressure sensor, and preferably a plurality of pressure sensors may be formed in an N×M matrix. This is for area segmentation in the step of performing area segmentation (S113), which will be described later, in order to generate a temperature distribution map in the present disclosure. Here, even if only one pressure sensor is used, body temperature changes in the user's cervical vertebral area can be monitored, but a plurality of pressure sensors may be used to obtain more accurate monitoring results by measuring body temperature changes according to the location of the cervical vertebral area.

In the step of determining whether the cervical vertebra is recognized (S111), it may be determined that the user's cervical vertebral area is recognized when a pressure greater than a preset level is measured through a pressure sensor. In addition, in another embodiment, the step of determining whether the cervical vertebra is recognized (S111) may be set to determine that the user's cervical vertebral area is recognized when a temperature in a preset temperature range is measured using a temperature sensor included in the monitoring method 10 of the present disclosure.

The step of performing area segmentation (S113) may be configured to perform segmentation on a cervical vertebra recognition area, which is an area where the cervical vertebra is recognized in the step of determining whether the cervical vertebra is recognized (S111). In the step of performing area segmentation (S113), the area where the cervical vertebra is recognized may be acquired using a pressure sensor, and at least two segmented areas may be generated by performing segmentation on the pertinent area.

Here, the segmented areas may be formed by using a preset number of or a preset reference wideness of segmented areas, and each of the segmented areas may be formed to include at least one temperature sensor. In this case, in the monitoring method 10 of the present disclosure, temperature changes for the respective segmented areas may be acquired.

When a plurality of segmented areas are acquired through segmentation of the cervical vertebra recognition area, the temperature distribution analysis performing step (S13) may be configured to measure the body temperature of the cervical vertebra recognition area in the temperature distribution analysis unit using at least one temperature sensor and generate a temperature distribution map using the measurement result. To this end, as shown in FIG. 7, the temperature distribution analysis performing step (S13) according to an embodiment of the present disclosure may be configured to include the steps of: acquiring body temperature distribution information (S131) and generating a temperature distribution map (S133).

The step of acquiring body temperature distribution information (S131) may be configured to acquire body temperature distribution information, which is a result of measuring the user's body temperature using a temperature sensor provided in the cervical vertebra recognition area. The step of acquiring body temperature distribution information (S131) may be configured to acquire body temperatures using a temperature sensor. Here, as described above, at least one temperature sensor may be included in the segmented areas, and by using this, in the step of acquiring body temperature distribution information (S131), the body temperature distribution information, which is a body temperature measurement result for each segmented area.

The step of generating a temperature distribution map (S133) may be configured to generate a temperature distribution map for the cervical vertebra recognition area using the segmented areas and body temperature distribution information. The temperature distribution map may be generated using the segmented areas, and may be formed to express the body temperature distribution information of the entire cervical vertebra recognition area in two dimensions using the body temperature distribution information measured in each of the segmented areas.

Meanwhile, the step of generating a temperature distribution map (S133) may be configured to perform defining on each of the segmented areas using machine learning. When the user's cervical vertebra is recognized, step of generating a temperature distribution map (S133) may be configured to acquire the distribution of measured pressure values and use the same as learning data, while defining the positions of segmented areas constituting the cervical vertebra recognition area of a current user by applying the previously obtained distribution of pressure values of other users to a preset algorithm.

When the generation of the temperature distribution map is completed, the body temperature change measurement step (S15) may be configured to derive a treatment effect by using the temperature distribution map. To this end, the body temperature change measurement step (S15) may be configured to include the steps of: generating a profile for each segmented area (S151) and analyzing a treatment effect (S153), as shown in FIG. 8.

The step of generating a profile for each segmented area (S151) may be configured to generate a profile for each segmented area by cumulatively storing the temperature distribution map. Here, the profile for each segmented area may cumulatively store temperature distribution maps, and thus may include time-series information. The profile for each segmented area may be generated including at least the temperature distribution map before treatment and the temperature distribution map after treatment. More preferably, the profile for each segmented area may be generated including all temperature distribution maps that are cumulatively stored at a preset cycle using the body temperature measured from the moment the recognition of the user's cervical vertebra is performed until just before the recognition of the user's cervical vertebra is completed.

The step of analyzing a treatment effect (S153) may be configured to derive a treatment effect by analyzing the profile for each segmented area. Here, the treatment effect may be derived by deriving a body temperature change for each of the segmented areas and comparing the derived body temperature change with a preset reference. In an embodiment of the present disclosure, in the step of analyzing a treatment effect (S153), a reference temperature distribution map among a plurality of temperature distribution maps included in the profile for each segmented area may be selected to derive the body temperature changes, and the amount of temperature change in each segmented area may be obtained as time-series information based on the temperature of the selected standard temperature distribution map.

In addition, the step of analyzing a treatment effect (S153) may be configured to further derive a personal treatment effect for an actual pertinent user by comparing the obtained treatment effect with an expected effect. In the step of analyzing a treatment effect (S153), the expected effect corresponding to a treatment method that is pre-entered by a treatment practitioner may be obtained and the obtained expected effect may be compared with the treatment effect to derive how effective the treatment method actually is for a pertinent user. Here, the actual effect can be expressed as a percentage, which is the ratio of the treatment effect to the expected effect.

The step of analyzing a treatment effect (S153) may be configured to derive treatment effects using, in addition to time-series information, a method for analyzing other profiles for the segmented areas. To derive a treatment effect for a specific portion, in the step of analyzing a treatment effect (S153), the treatment effect may be derived by comparing the left and right profiles of a cervical vertebral area for each segmented area, and, to determine how effective the personal treatment effect for the pertinent user is, in the step of analyzing a treatment effect (S153), a comparison with other users' profiles for each segmented area may be performed. Here, in the step of analyzing a treatment effect (S153), the extent of the effect of the treatment provided to the pertinent user may be relatively determined by cumulatively storing the treatment effects for other users, and an optimal treatment method may be identified and provided using the profiles of other users showing similar treatment effects for each segmented area.

According to the present disclosure, the effect of treatment performed on the user can be accurately identified by cumulatively storing and comparing body temperature changes in the cervical area.

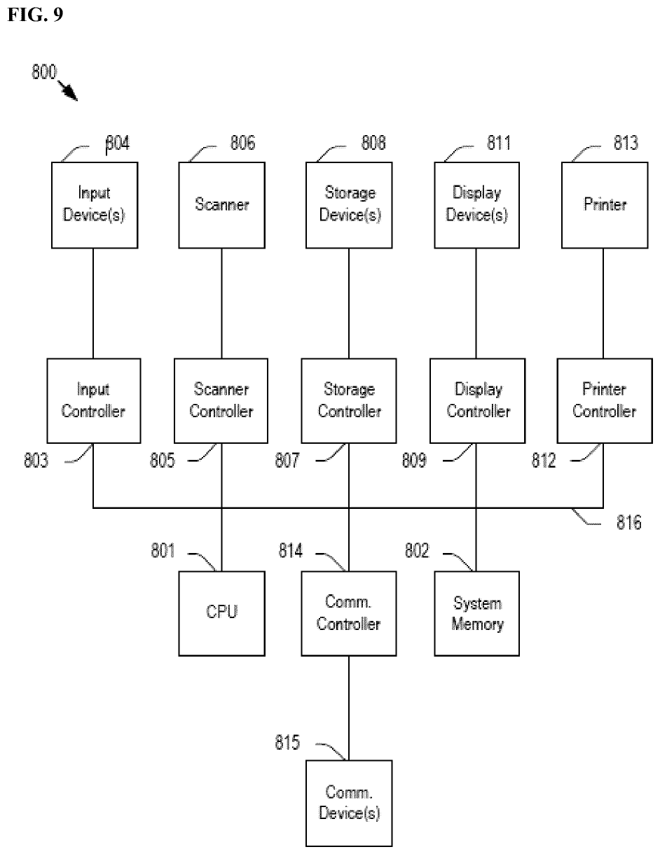

FIG. 9 shows a schematic diagram of a system for implementing one or more aspects of the present disclosure. It will be understood that the functionalities shown for system 800 may operate to support various embodiments shown in FIGS. 1 to 4—although it shall be understood that the system may be differently configured and include different components. As illustrated in FIG. 9, the system 800 may include a central processing unit (CPU) 801 that provides computing resources and controls the computer. CPU 801 may be implemented with a microprocessor or the like, and may also include a graphics processor and/or a floating-point coprocessor for mathematical computations. System 800 may also include a system memory 802, which may be in the form of random-access memory (RAM) and read-only memory (ROM).

A number of controllers and peripheral devices may also be provided, as shown in FIG. 9. An input controller 803 represents an interface to various input device(s) 804, such as a keyboard, mouse, or stylus. There may also be a scanner controller 805, which communicates with a scanner 806. System 800 may also include a storage controller 807 for interfacing with one or more storage devices 808 each of which includes a storage medium such as magnetic tape or disk, or an optical medium that might be used to record programs of instructions for operating systems, utilities and applications which may include embodiments of programs that implement various aspects of the present invention. Storage device(s) 808 may also be used to store processed data or data to be processed in accordance with the invention. System 800 may also include a display controller 809 for providing an interface to a display device 811, which may be a cathode ray tube (CRT), a thin film transistor (TFT) display, or other type of display. System 800 may also include a printer controller 812 for communicating with a printer 813. A communications controller 814 may interface with one or more communication devices 815, which enables system 800 to connect to remote devices through any of a variety of networks including the Internet, an Ethernet cloud, an FCoE/DCB cloud, a local area network (LAN), a wide area network (WAN), a storage area network (SAN) or through any suitable electromagnetic carrier signals including infrared signals.

In the illustrated system, all major system components may connect to a bus 816, which may represent more than one physical bus. However, various system components may or may not be in physical proximity to one another. For example, input data and/or output data may be remotely transmitted from one physical location to another. In addition, programs that implement various aspects of this invention may be accessed from a remote location (e.g., a server) over a network. Such data and/or programs may be conveyed through any of a variety of machine-readable medium including, but are not limited to: magnetic media such as hard disks, floppy disks, and magnetic tape; optical media such as CD-ROMs and holographic devices; magneto-optical media; and hardware devices that are specially configured to store or to store and execute program code, such as application specific integrated circuits (ASICs), programmable logic devices (PLDs), flash memory devices, and ROM and RAM devices. Embodiments of the present invention may be encoded upon one or more non-transitory computer-readable media with instructions for one or more processors or processing units to cause steps to be performed. It shall be noted that the one or more non-transitory computer-readable media shall include volatile and non-volatile memory. It shall be noted that alternative implementations are possible, including a hardware implementation or a software/hardware implementation. Hardware-implemented functions may be realized using ASIC(s), programmable arrays, digital signal processing circuitry, or the like. Accordingly, the “means” terms in any claims are intended to cover both software and hardware implementations. Similarly, the term “computer-readable medium or media” as used herein includes software and/or hardware having a program of instructions embodied thereon, or a combination thereof. With these implementation alternatives in mind, it is to be understood that the figures and accompanying description provide the functional information one skilled in the art would require to write program code (i.e., software) and/or to fabricate circuits (i.e., hardware) to perform the processing required.

It shall be noted that embodiments of the present disclosure may further relate to computer products with a non-transitory, tangible computer-readable medium that have computer code thereon for performing various computer-implemented operations. The media and computer code may be those specially designed and constructed for the purposes of the present invention, or they may be of the kind known or available to those having skill in the relevant arts. Examples of tangible computer-readable media include, but are not limited to: magnetic media such as hard disks, floppy disks, and magnetic tape; optical media such as CD-ROMs and holographic devices; magneto-optical media; and hardware devices that are specially configured to store or to store and execute program code, such as application specific integrated circuits (ASICs), programmable logic devices (PLDs), flash memory devices, and ROM and RAM devices. Examples of computer code include machine code, such as produced by a compiler, and files containing higher level code that are executed by a computer using an interpreter. Embodiments of the present invention may be implemented in whole or in part as machine-executable instructions that may be in program modules that are executed by a processing device. Examples of program modules include libraries, programs, routines, objects, components, and data structures. In distributed computing environments, program modules may be physically located in settings that are local, remote, or both.

One skilled in the art will recognize no computing system or programming language is critical to the practice of the present invention. One skilled in the art will also recognize that a number of the elements described above may be physically and/or functionally separated into sub-modules or combined together.

The foregoing embodiment is merely an exemplary explanation of the inventive concept of the present disclosure. A person skilled in the art would make various modifications and variations without departing from the essential characteristics of the present disclosure.

Accordingly, the embodiments disclosed in the present disclosure are not intended to limit, but rather to explain, the inventive concept of the present disclosure. In addition, the scope of the inventive concept of the present disclosure is not limited by the embodiments. The scope of protection of the present disclosure should be interpreted in accordance with the appended claims, and all inventive concepts within the equivalent scope should be interpreted as being included in the scope of rights of the present disclosure.

What is claimed is:

1. A cervical body temperature change monitoring system comprising:
a cervical vertebral area processing unit that determines whether the cervical vertebra is recognized using at least one pressure sensor and performs segmentation on cervical vertebra recognition area to generate a plurality of segmented areas;
a temperature distribution analysis unit that measures a body temperature in the cervical vertebra recognition area using at least one temperature sensor and generates a temperature distribution map using the measurement results; and
a body temperature change measurement unit that derives a treatment effect by using the temperature distribution map.

2. The cervical body temperature change monitoring system as claimed in claim 1, wherein the cervical vertebral area processing unit comprises:
a cervical vertebra recognition determination module that determines whether the cervical vertebra is recognized using the pressure sensor; and
an area segmentation module that, when the user's cervical vertebra is recognized, generates at least two segmented areas by performing segmentation on the cervical vertebra recognition area, which is an area in which the cervical vertebra is recognized.

3. The cervical body temperature change monitoring system as claimed in claim 2, wherein the segmented areas are formed by using a preset number of or a preset reference wideness of segmented areas, and each of the segmented areas is formed to include at least one temperature sensor.

4. The cervical body temperature change monitoring system as claimed in claim 1, wherein the temperature distribution analysis unit comprises:
a body temperature information acquisition module that acquires body temperature distribution information as a measurement result of the body temperature using the temperature sensor provided in the cervical vertebra recognition area; and
a temperature distribution map generation module that generates the temperature distribution map for the cervical vertebra recognition area using the segmented areas and the body temperature distribution information.

5. The cervical body temperature change monitoring system as claimed in claim 4, wherein the temperature distribution map is generated using the segmented areas and the body temperature distribution information, and includes a definition for each of the segmented areas through machine learning.

6. The cervical body temperature change monitoring system as claimed in claim 1, wherein the body temperature change measurement unit comprises:
a segmented area profile generation module that cumulatively stores the temperature distribution map to generate a profile for each segmented area; and
a treatment effect analysis module that derives the treatment effect by analyzing the profile for each segmented area.

7. The cervical body temperature change monitoring system as claimed in claim 6, wherein the profile for each segmented area is generated by using the temperature distribution map from the moment the body temperature is first measured to the moment the treatment is completed.

8. The cervical body temperature change monitoring system as claimed in claim 7, wherein the treatment effect is derived by deriving the change in the body temperature for each of the segmented areas and comparing the derived change in the body temperature with a preset reference.

9. A cervical body temperature change monitoring method comprising:
a cervical vertebral area processing step of a cervical vertebral area processing unit determining whether the cervical vertebra is recognized using at least one pressure sensor and performing segmentation on a cervical vertebra recognition area to generate a plurality of segmented areas;
a temperature distribution analysis performing step of a temperature distribution analysis unit measuring a body temperature in the cervical vertebra recognition area using at least one temperature sensor and generating a temperature distribution map using the measurement results; and
a body temperature change measurement performing step of a body temperature change measurement unit deriving a treatment effect by using the temperature distribution map.

10. The cervical body temperature change monitoring method as claimed in claim 9, wherein the cervical vertebral area processing step comprises:
determining whether the cervical vertebra is recognized using the pressure sensor; and
when the user's cervical vertebra is recognized, generating at least two segmented areas by performing segmentation on the cervical vertebra recognition area, which is an area in which the cervical vertebra is recognized.

11. The cervical body temperature change monitoring method as claimed in claim 10, wherein the segmented areas are formed by using a preset number of or a preset reference wideness of segmented areas, and each of the segmented areas is formed to include at least one temperature sensor.

12. The cervical body temperature change monitoring method as claimed in claim 9, wherein the temperature distribution analysis performing step comprises:
acquiring body temperature distribution information as a measurement result of the body temperature using the temperature sensor provided in the cervical vertebra recognition area; and
generating the temperature distribution map for the cervical vertebra recognition area using the segmented areas and the body temperature distribution information.

13. The cervical body temperature change monitoring method as claimed in claim 12, wherein the temperature distribution map is generated using the segmented areas and the body temperature distribution information, and includes a definition for each of the segmented areas through machine learning.

14. The cervical body temperature change monitoring method as claimed in claim 9, wherein the body temperature change measurement performing step comprises:
cumulatively storing the temperature distribution map to generate a profile for each segmented area; and
deriving the treatment effect by analyzing the profile for each segmented area.

15. The cervical body temperature change monitoring method as claimed in claim 14, wherein the profile for each segmented area is generated by using the temperature distribution map from the moment the body temperature is first measured to the moment the treatment is completed.

16. The cervical body temperature change monitoring method as claimed in claim 15, wherein the treatment effect is derived by deriving the change in the body temperature for each of the segmented areas and comparing the derived change in the body temperature with a preset reference.
